(19) United States
(12) Patent Application Publication
(10) Pub. No.: US 2011/0165122 A1
(43) Pub. Date: Jul. 7, 2011

(54) METHOD FOR TARGETED AND SUSTAINED ANTIVIRAL THERAPY

(75) Inventors: Arash Shahangian, Los Angeles, CA (US); Genhong Cheng, Calabassas, CA (US); Lucy S. Cheng, Calabassas, CA (US); Kham Moc Trinh, Alhambra, CA (US); Paul W. Dempsey, Studio City, CA (US); Beichu Guo, Arcadia, CA (US); Sherie L. Morrison, Los Angeles, CA (US)

(73) Assignee: The Regents of the University of California, Oakland, CA (US)

(21) Appl. No.: 12/943,757

(22) Filed: Nov. 10, 2010

Related U.S. Application Data
(60) Provisional application No. 61/259,965, filed on Nov. 10, 2009.

(51) Int. Cl. A61K 38/21; A61K 39/395; A61P 31/12; A61P 37/02; A61P 11/06; A61P 3/10; A61P 37/08; A61P 19/02

(52) U.S. Cl. 424/85.6; 424/134.1; 424/85.7; 424/85.4

(57) ABSTRACT

Compounds compositions and methods of modulating the immune response are provided. The method uses fusion proteins of a cytokine and an antibody to potentiate the action of the cytokine.

### CROSS-REFERENCES TO RELATED APPLICATIONS

This application claims priority benefit of U.S. Provisional Application Ser. No. 61/259,965, filed Nov. 10, 2009, which is incorporated by reference in its entirety for all purposes.

### STATEMENT AS TO RIGHTS TO INVENTIONS MADE UNDER FEDERALLY SPONSORED RESEARCH AND DEVELOPMENT

This work was supported by NIH T32 AI007323-19 and GM 08042 (A.S.), and NIH 1R01A1056154 & 1R01A1069120 (G.C.). The U.S. Government has certain rights in this invention.

### REFERENCE TO A “SEQUENCE LISTING,” A TABLE, OR A COMPUTER PROGRAM LISTING APPENDIX SUBMITTED IN COMPUTER READABLE FORM

NOT APPLICABLE

### FIELD OF THE INVENTION

This invention relates to methods of anti-viral therapy using cytokines linked to antibodies.

### BACKGROUND OF THE INVENTION

Interferons were first described for their ability to protect cells from viral infections¹. Since these initial reports, three major types of interferons (IFNs) have been described. Type I IFNs, including IFNβ and multiple IFNα subtypes, induce antiviral gene programs through the IFNα/β receptor, IFNAR. Many of these genes directly and indirectly are involved in curbing viral replication and spread². Type I IFNs have been used to treat both chronic viral infections such as those with hepatitis B and C, and also a variety of neoplastic conditions such as melanoma, hairy cell leukemia, and non-Hodgkin's lymphoma³⁻⁷. Recently, PEGylated IFNs, which show decreased clearance as compared to recombinant interferons have emerged as the standard of care. While PEGylation has increased the therapeutic efficacy of IFN therapy, a wide variety of unpleasant and serious side-effects remain. In this study, we set out to determine whether antibody-IFN fusions could be used as an alternative and allow for specific targeting of IFNs which we believe would be beneficial in reducing undesired side-effects.

IFN-β, a type I IFN, is widely used for the treatment of MS. However, the mechanisms behind its therapeutic efficacy are not well understood. Using a murine model of MS, EAE, we demonstrated that the Th17-mediated development of autoimmune disease is constrained by Toll-IL-1 receptor domain-containing adaptor inducing IFN-β-dependent (TRIF-dependent) type I IFN production and its downstream signaling pathway. Mice with defects in TRIF or type I IFN receptor (IFNAR) developed more severe EAE. Notably, these mice exhibited marked CNS inflammation, as manifested by increased IL-17 production. In addition, IFNAR-dependent signaling events were essential for negatively regulating Th17 development. Finally, IFN-β-mediated IL-27 production by innate immune cells was critical for the immunoregulatory role of IFN-β in the CNS autoimmune disease. Together, our findings not only may provide a molecular mechanism for the clinical benefits of IFN-β in MS but also demonstrate a regulatory role for type I IFN induction and its downstream signaling pathways in limiting Th17 development and autoimmune inflammation (see, Guo et al., J. Clin. Invest. 118:1680-1690 (2008)).

Given the importance of cytokines in mediating the immune response and defenses against infectious organisms, there is a need for advantageous treatments which modulate such responses. The present invention provides for these and other methods by providing antibody-cytokine fusion proteins, their pharmaceutical compositions and methods of treatment based upon the use of such modified cytokines.

### BRIEF SUMMARY OF THE INVENTION

This invention provides a novel approach to the treatment of viral infections and modulation of the immune response. In a first aspect, the invention pertains to the discovery that a chimeric molecule comprising a cytokine and an antibody provides a highly effective agent for the treatment of a viral infection. Thus in one embodiment, this invention provides a construct comprising a cytokine fused to an antibody. In human applications, the antibody and cytokine are preferably human. In particularly preferred embodiments the cytokine is covalently attached to the antibody at a carboxyl terminus of the antibody or fragment thereof. The antibody and cytokine may be connected by a linker. In one embodiment, the fusion protein is chemically constructed or recombinantly expressed. In such fusion proteins, the antibody and cytokine are directly joined, or more preferably, joined by a peptide linker ranging in length from 2 to about 50, more preferably from about 2 to about 20, and most preferably from about 2 to about 10 amino acids.

In another embodiment this invention provides a composition comprising the chimeric molecules described herein and a pharmaceutically acceptable diluent or excipient.

The invention also provides a nucleic acid (e.g. a DNA or an RNA) encoding a fusion protein comprising a cytokine and an antibody or fragment thereof as described herein. The nucleic acid is preferably in an expression cassette and in certain embodiments, the nucleic acid is present in a vector (e.g. a baculoviral vector). This invention also provides a host cell transfected with one or more of the nucleic acids described herein. The host cell is preferably a eukaryotic cell, including mammalian (e.g., mouse, rat, human) and insect cells.

In another embodiment, this invention provides methods of treating a viral infection in a subject by promoting the action of a cytokine useful in fighting the infection. The methods involve targeting and/or stabilizing the cytokine by coupling it to an antibody targeting an infected cells or cells of a tissue known to be susceptible to infection by the virus. The antibodies may target a viral epitope or a tissue-specific antigen. The subject may be a mammal, a primate, or a human. The infected cell can be any cell within the subject (e.g., epithelial cell, endothelial cell, mesothelial cells, adipocytes, nerve cells, lymphocytes, hepatocytes, B-cells, fibroblasts) or a cell of any tissue, organ, or organ system of a subject (e.g., bone, muscle, lung, liver, kidney, pancreas, esophagous, GI tract, respiratory tract, skin, mucosa, eye, immune system, nervous system, nerve cell, endocrine or exocrine cell, pancreas, reproductive system, bone marrow).

In another aspect, the invention provides a method of modulating the immune system by coupling a cytokine which modulates the activity of a cell of the immune system to an antibody which binds to that immune system cell. The invention also provides the corresponding fusion proteins and nucleic acids encoding the fusion protein. In some embodiments, the invention provides methods of activating or inhibiting the immune system in the treatment of disease.

In another aspect, the invention provides antibodies, and fusion proteins of the antibodies. In other embodiments this invention provides a composition comprising the chimeric molecules described herein and a pharmaceutically acceptable diluent or excipient.

Accordingly, in some embodiments, the invention provides a method of treating a virus infection in a subject by administering a fusion protein to the subject, wherein the fusion protein comprises a cytokine fused to an antibody or a fragment of the antibody which binds to a cell infected by the virus. For instance, in a method of treating a hepatitis B or C infection in a human, the cell can be a hepatocyte and the antibody can bind to a cell surface antigen of the hepatocytes and the cytokine can be a human interferon α or interferon β or a hybrid interferon. In some other embodiments, the subject is human. In one particular embodiment, the cytokine is fused to a C-terminus of the antibody. The antibody can contain a CDR determining sequence of an antibody which recognizes the target cell. The antibody can be an scFv fragment, a diabody, a minibody, a triabody which can bind to a target cell or tissue. In some embodiments, of the above, the virus is HCV, HBV, HSV, HPV, or HIV and the subject is human. In some embodiments, the antibody or fragment thereof recognizes an epitope of the virus or another constituent of the cell surface of a target cell. In some embodiments, the antibody is an anti-HBV antibody that can target an IFN fused to it to virally infected cells such as an hepatocyte. Accordingly, in some embodiments, it is envisioned that the target cell is a cell bearing a cell surface antigen of the virus.

In some other embodiments of the second aspect, the invention also provides a method of modulating an immune response in a subject, said method comprising administering to the subject a fusion protein comprising a cytokine to an antibody which binds to a target cell of the immune system. In some embodiments, the cytokine is an interferon and the target cell is a Th17 cell of the mouse or the human equivalent. In preferred embodiments, an autoimmune condition of the subject is treated. Examples of autoimmune conditions include, but are not limited to, Multiple sclerosis, myasthenia gravis, asthma, allergy, IBD or colitis, rheumatoid arthritis, Graves disease, or Type I diabetes. In preferred embodiments, the target immune cell is a Treg cell or its precursor. The subject can be a mammal (e.g., mouse, rabbit, rat, primate), preferably a human.

### BRIEF DESCRIPTION OF THE DRAWINGS

FIG. 1. (a) 38C13 cells were stimulated with IgG3-IFNα for 30 minutes, and phosphorylation of STAT1 was assayed by western blot. (b) IgG3-IFNα protects cells from viral infection more effectively than IFN across a wide range of doses. Anti-HER2-IgG3-IFNα offered better antiviral protection than IgG3-IFNα. Cells were infected with MHV-68-Luc and treated with indicated reagents. Cells were harvested 48 hours post infection and assayed for luciferase activity. (c) IgG-IFNα inhibits expression of luciferase better than IFNα alone in nasal passages of MHV-68-luc infected mice. Mice were infected with 5000 PFU of virus and treated with indicated reagents i.p. Bioluminescence was measured on day 5 post infection. (d) Bioluminescence from nasal passages of IgG3-IFNα treated animals a showed statistically significant reduction in luciferase expression, as compared to IFN group in all measured planes (supine and one lateral plane are graphed). (e) On day 7 post infection mice were sacrificed and lung homogenates were assayed for viral load by plaque assays and Q-PCR. MHV-68 viral titers are effectively reduced in the lungs of IgG3-IFN treated animals as compared to those treated with IFNα or IgG alone.

FIG. 2. Murine NIH 3T3 or human 293T cells were stimulated with the indicated numbers of units for 30 minutes at 37° C. Whole cell Ripa lysates were prepared and probed for phospho-STAT1 or total STAT1 levels as indicated.

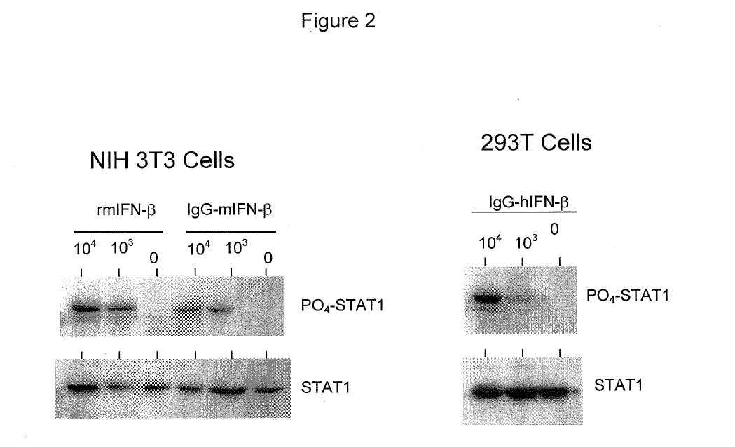

FIG. 3. Vector and nucleic acid and sequence information for expressing a fusion protein according to the invention.

FIG. 4. Sequence of a heavy chain HBV-specific antibody for use according to the invention.

FIG. 5. Sequence of a light chain HBV-specific antibody sequence for use according to the invention.

FIG. 6. Sequence of a heavy chain HIV-specific antibody sequence for use according to the invention.

### DETAILED DESCRIPTION OF THE INVENTION

This invention relates to the surprising finding that fusion of Type I interferons to immunoglobulin has three effects that are desirable for the biological applications of the interferon activity. Firstly, fusion of interferon to IgG molecules increases the potency of the interferon activity. Secondly, fusion of interferon to IgG molecules makes interferon more stable. Thirdly, fusion of interferon to IgG molecules allows targeting of interferon to specific structures using the antigen binding domain of the IgG fusion partner. All of these aspects, alone or in combination, increase the potency, stability and specificity while decreasing the side effects of interferon.

Fusion of interferon-α with IgG has been shown to increase the potency of the antiviral affect of the interferon domain by in vitro assays. Importantly, fusion of interferon-α or interferon-β to IgG does not inhibit the ability of interferon to bind to the Type I interferon Receptor. Therefore, interferon potency is increased by an order of magnitude by fusion to IgG molecules. This higher potency will allow for decreased dose requirements for clinical applications.

In a second example, fusion of interferon-α with IgG has been shown to increase the antiviral effects by in vivo assays, which require both the stability and potency of the fusion protein. Recombinant IFN therapy is faced with several important challenges. rIFNs are rapidly cleared from the body through the kidneys with the average half life of 2.5 hours. This necessitates repetitive administration of IFN therapy. Fusion of interferon-α or interferon-β to IgG significant increases the stability of interferon.

In a third example, fusion of interferon-α with antigen specific IgG has been shown to dramatically increase the efficiency of targeting interferon to specific cells of interest. The type I interferon is ubiquitously expressed in most cells in the body. A major problem with the current treatments of interferon-α and interferon-β is the unwanted side effects.

In particular applications, for instance, interferon-α or interferon-β can be targeted to cells infected with virus using IgG variable regions that target the cytokine to the appropriate cell. In one embodiment, replacement of the anti-DNS variable region sequence with the antigen binding domain of an anti-HBV or anti-HIV hybridoma is contemplated. This targeting affords directing the biological activity of the interferon to cells that are infected with a specific virus. Again this has the impact of reducing the amount of interferon that needs to be administered with a resulting decrease in the number and severity of the side effects. We have already demonstrated that interferon-α fused to IgG has increased potency of anti-viral activity. In addition, the antiviral activity of the IgG fusion protein displays an even greater potency when targeted to an infected cell. Targeting interferon to Her2-neu expressing cells increases the potency of interferon-a by greater than one order of magnitude.

In another application, interferon fusion proteins will be used in an anti-inflammatory role. Treatment with interferon effects an anti-inflammatory affect in a mouse model of multiple sclerosis by inhibiting development of Th17 cells. Interferon induces expression of IL-27 and thus constrains IL-17 expression. The impact of an antibody fusion protein or IgG-interferon fusion protein would be to similarly inhibit the development of EAE by decreasing the amount of Th17 activation. This mechanism of regulation would provide a broader anti-inflammatory application for antibody-interferon therapeutics, including asthma, allergy, IBD or colitis, rheumatoid arthritis and myasthenia gravis.

Cytokines for use in any aspect of the invention include, but are not limited to, the interleukins, chemokines, lymphokines, monokines, and interferons (e.g., interferons α,β, γ, and δ) and TNF-α. The cytokine can be a proinflammatory or anti-inflammatory cytokine (IL-1, TNF-alpha)·Th1 (interferon-gamma and tumor necrosis factor-beta), Th2 (interleukin 4, interleukin 5, interleukin 6, interleukin 10, interleukin 13) or anti-inflammatory cytokine with respect to the targeted immune response or cell. Contemplated cytokines for use in the methods and compositions of the invention include, but are not limited to, GM-CSF, IL-1 alpha, IL-1 beta, IL-2, IL-3, IL-4, IL-5, IL-6, IL-7, IL-8, IL-10, IL-12, IFN-alpha, IFN-beta, IFN-gamma, MIP-1 alpha, MIP-1 beta, TGF-beta, TNF-alpha, and TNF-beta. The fusion protein may be pegylated. The cytokine may represent a peptide fragment of the cytokine having the biological activity of the full cytokine. Preferred cytokines are of human origin and/or having a sequence at least 90%, 95%, 96%, 97%, 98%, 99%, or 100% identical to a human sequence of the same length.

Viruses targeted by the invention include, for instance, mammalian viruses including viruses which can infect humans such as hepatitis C virus (HCV), hepatitis B virus (HBV), herpes simplex virus (HSV), human papilloma virus (HPV), human immunodeficiency virus (HIV), cytomegalovirus (CMV), and Epstein-Barr virus (EBV), yellow fever. Ebola, Western Equine and Nile encephalitis viruses, smallpox, shingles, and hemorrhagic fever viruses.

A “fusion protein” refers to a polypeptide formed by the joining of two or more polypeptides through a peptide bond formed between the amino terminus of one polypeptide and the carboxyl terminus of another polypeptide. The fusion protein may be formed by the chemical coupling of the constituent polypeptides, or it may be expressed as a single polypeptide from nucleic acid sequence encoding the single contiguous fusion protein. A single chain fusion protein is a fusion protein having a single contiguous polypeptide backbone.

A “spacer” or “linker” as used in reference to a fusion protein refers to a peptide or amino acid that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, susceptibility to enzyme cleavage by intracellular or extracellular enzymes, including tissue-specific enzymes, or hydrophobicity of the molecule. Linkers, if present, can be preferably from 1 to 15 amino acids in length, 1 to 10 amino acids in length, or 2 to 5 amino acids in length.

Using the known cytokine sequence information nucleic acids encoding the interferons and the selected antibody or fragment thereof, a chimeric cytokine fusion antibody can be produced using standard methods well known to those of skill in the art. For example, the nucleic acid(s) may be cloned, or amplified by in vitro methods, such as the polymerase chain reaction (PCR), the ligase chain reaction (LCR), the transcription-based amplification system (TAS), the self-sustained sequence replication system (SSR), etc. A wide variety of cloning and in vitro amplification methodologies are well known to persons of skill in the art.

Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864.

In a particularly preferred embodiment, the chimeric molecules of this invention are fusion proteins. The fusion protein can be chemically synthesized using standard chemical peptide synthesis techniques, or, more preferably, recombinantly expressed. Where both molecules are relatively short the chimeric molecule may be synthesized as a single contiguous polypeptide. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963), and Stewart et al., Solid Phase Peptide Synthesis, 2nd ed. Pierce Chem. Co., Rockford, Ill. (1984).

In a most preferred embodiment, the chimeric fusion proteins of the present invention are synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion protein of this invention may be prepared by any suitable method, including, for example, cloning and restriction of appropriate sequences or direct chemical synthesis by methods such as the phosphotriester method of Narang et al Meth. Enzymol. 68: 90-99 (1979); the phosphodiester method of Brown et al., Meth. Enzymol. 68: 109-151 (1979); the diethylphosphoramidite method of Beaucage et al., Tetra. Lett., 22: 1859-1862 (1981); and the solid support method of U.S. Pat. No. 4,458,066.

Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

Alternatively, subsequences may be cloned and the appropriate subsequences cleaved using appropriate restriction enzymes. The fragments may then be ligated to produce the desired DNA sequence.

Antibodies for use according to any aspect of the invention include, but are not limited to, recombinant antibodies, polyclonal antibodies, monoclonal antibodies, chimeric antibodies, human monoclonal antibodies, humanized or primatized monoclonal antibodies, and antibody fragments. The antibodies preferably bind to an external loop or sequence of cell surface protein. “Antibody” accordingly refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. The IgG class is exemplified herein.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Accordingly, the term antibody also embraces minibodies, diabodies, triabodies and the like. Diabodies are small bivalent biospecific antibody fragments with high avidity and specificity. Their high signal to noise ratio is typically better due to specificity and fast blood clearance increasing their potential for diagnostic and therapeutic targeting of specific antigen (Sundaresan et al., J Nucl Med 44:1962-9 (2003). In addition, these antibodies are advantageous because they can be engineered if necessary as different types of antibody fragments ranging from a small single chain Fv to an intact IgG with varying isoforms (Wu & Senter, Nat. Biotechnol. 23:1137-1146 (2005)). In some embodiments, the antibody fragment is part of a diabody. In some embodiments, the invention provides high avidity antibodies for use according to the invention.

The CDR regions of an antibody may be used to construct a binding protein, including without limitation, an antibody, a scFv, a triabody, a diabody, a minibody, and the like. In a certain embodiment, a binding protein of the invention will comprise at least one or all the CDR regions from an antibody. CDR sequences may be used on an antibody backbone, or fragment thereof, and likewise may include humanized antibodies, or antibodies containing humanized sequences. Methods of identifying CDR portions of an antibody are well known in the art. See, Shirai, H., Kidera, A., and Nakamura, H., H3-rules: Identification of CDR-H3 structures in antibodies, FEBS Lett., 455(1):188-197, 1999; and Almagro J C, Fransson, J. Front Biosci. 13:1619-33 (2008). In some embodiments, the antibody sequence comprises as CDR sequence of an antibody sequence provided herein.

Diabodies, first described by Hollinger et al., PNAS (USA) 90(14): 6444-6448 (1993), may be constructed using heavy and light chains disclosed herein, as well as by using individual CDR regions disclosed herein. Typically, diabody fragments comprise a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{H} and V_{L} domains of another fragment, thereby forming two antigen-binding sites. Triabodies can be similarly constructed with three antigen-binding sites. An Fv fragment contains a complete antigen-binding site which includes a V_{L} domain and a V_{H} domain held together by non-covalent interactions. Fv fragments embraced by the present invention also include constructs in which the V_{H} and V_{L} domains are crosslinked through glutaraldehyde, intermolecular disulfides, or other linkers. The variable domains of the heavy and light chains can be fused together to form a single chain variable fragment (scFv), which retains the original specificity of the parent immunoglobulin. Single chain Fv (scFv) dimers, first described by Gruber et al., J. Immunol. 152(12):5368-74 (1994), may be constructed using heavy and light chains disclosed herein, as well as by using individual CDR regions disclosed herein. Many techniques known in the art can be used to prepare the specific binding constructs of the present invention (see, U.S. Patent Application Publication No. 20070196274 and U.S. Patent Application Publication No. 20050163782, which are each herein incorporated by reference in their entireties for all purposes, particularly with respect to minibody and diabody design).

Bispecific antibodies can be generated by chemical cross-linking or by the hybrid hybridoma technology. Alternatively, bispecific antibody molecules can be produced by recombinant techniques (see: bispecific antibodies). Dimerization can be promoted by reducing the length of the linker joining the VH and the VL domain from about 15 amino acids, routinely used to produce scFv fragments, to about 5 amino acids. These linkers favor intrachain assembly of the VH and VL domains. A suitable short linker is SGGGS but other linkers can be used. Thus, two fragments assemble into a dimeric molecule. Further reduction of the linker length to 0-2 amino acids can generate trimeric (triabodies) or tetrameric (tetrabodies) molecules.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., in Monoclonal Antibodies and Cancer Therapy, Alan R. Liss, Inc., pp. 77-96 (1985); Coligan, Current Protocols in Immunology (1991); Harlow & Lane, Antibodies, A Laboratory Manual (1988); and Goding, Monoclonal Antibodies Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

### EXAMPLES

### Example 1

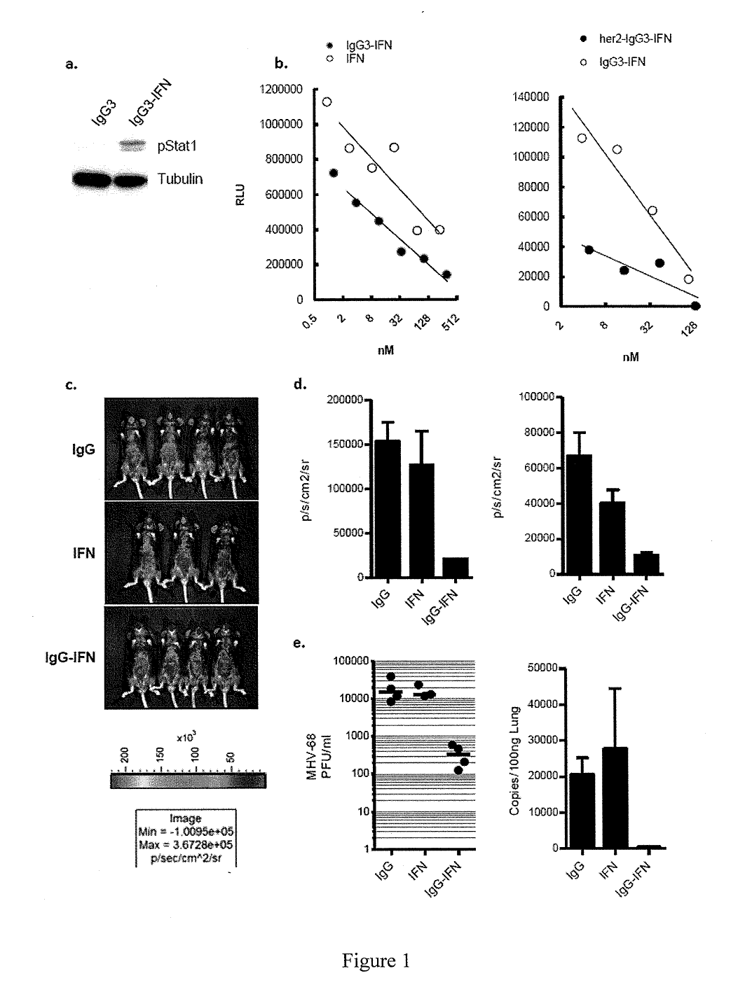

Use of chimeric molecules with murine IFNα fused to the carboxy-terminus of human IgG3 (IgG3-IFNα). As IFNα is also a potent antiviral cytokine, we examined the ability of IgG3-IFNα to activate antiviral pathway and inhibit viral replication. 38C13 cells stimulated with 1 μg of IgG3-IFNα for 60 minutes phosphorylated Stat1 (FIG. 1a). A recombinant MHV-68 virus (MHV-68-Luc) with the firefly luciferase gene under the viral M3 promoter integrated into the viral genome served as a convenient readout for viral replication in cultured cells and mice. To determine the ability of IgG3-IFNα to inhibit MHV-68 replication, 38C13 cells were infected with MHV-68-Luc and then treated with either IgG or IgG3-IFNα. IgG3-IFNα inhibited viral replication as measured by luciferase activity two days post infection (data not shown). To compare the relative antiviral efficiency of IgG3-IFNα with IFNα, 38C13 cells were infected with MHV-68-Luc virus and then treated with IFNα or IgG3-IFNα at the indicated concentrations. Luciferase activity of the cells was measured two days post infection. All experiments were performed in triplicate and repeated at least three times. As shown in FIG. 1b, IgG3-IFNα was more effective in inhibiting viral protein expression across a wide range of concentrations.

In addition to increasing the potency, a potential advantageous feature of antibody conjugated type I IFN is the possibility of using the antibody specificity to target type I IFN to specific cells. Therefore, we used anti-HER2-IgG3-IFNα, in which IFNα is fused to a HER2/neu-specific antibody, and 38C13 cells stably expressing the HER2/neu receptor (38C13-HER2). 38C13-HER2 cells infected with MHV-68-Luc were treated with IgG3-IFNα or anti-HER2-IgG3-IFNα following infection with MHV-68-Luc. MHV-68 luciferase activity was reduced more effectively following treatment with anti-HER2-IgG3-IFNα across a broad range of therapeutic doses (FIG. 1b, right panel). Importantly, this difference was more apparent with low concentrations, suggesting anti-HER2-IgG3-IFNα may increase effectiveness of IFNα by targeting IFNα to HER2/neu expressing cells at such concentrations. When parental 38C13 cells that did not express HER2/neu were used, both fusion proteins similarly inhibited viral replication (data not shown).

We used an intranasal model of infection with MHV-68-Luc virus, followed by bioluminescence imaging to determine the effectiveness of IgG3-IFNα in inhibiting viral replication in vivo. We first administrated 5000 PFU MHV-68-Luc through nasal passages and then treated intraperitoneally with 25,000 units IFNα, 25,000 units IgG3-IFNα (10 μg) or 10 μg IgG3 alone. Mice were imaged on day 5 (FIG. 1c). Bioluminescence readings of mice imaged in supine and lateral positions were obtained and are presented in the left and right panels of FIG. 1d. While mice treated with IFNα did exhibit a slight reduction in bioluminescence readings as compared to IgG treated animals, IgG3-IFNα-treated animals exhibited a statistically significant seven-fold reduction in readings obtained in either position (P<0.0001 as compared to IgG treated group). Indeed mice treated with IgG-IFN had significantly reduced bioluminescence readings as compared to IFNα treated mice (P<0.05).

On day 7 post infection with MHV-68, mice in each of the three groups were sacrificed, and the viral burden in the lungs was measured by plaque assay on lung homogenates. qPCR was also used to determine viral genome copy number in the lungs of infected animals. IgG3-IFNα treated animals exhibited a 100-fold reduction in viral burden as measured by plaque assay (p<0.05, Student's t-test). Surprisingly, treatment with IFNα provided no protection against viral burden as measured by this assay (FIG. 1e, left panel). Similarly, viral genomic content in the lungs of IgG3-IFNα-treated animals was 600 times lower than those observed in IgG-treated animals, while IFNα treatment seemed insufficient to reduce viral genomic burden (FIG. 1e, right panel). Thus, IgG3-IFNα proved to be a more potent antiviral agent both in vitro and in vivo.

### Example 2

Anti-DNS-IgG3-muIFNα long glyser linker-nucleic
acid sequence (SEQ ID NO: 1):
ATGTACTTGGGACTGAACTGTGTAATCATAGTTTTTCTCTTAAAAGGTG
TCCAGAGTGAAGTCAAGCTTGAGGAGTCTGGAGGAGGCTTGGTGCAACC
TGGAGGTTCCATGAAACTCTCTTGTGCTACTTCTGGATTCACTTTTAGT
GATGCCTGGATGGACTGGGTCCGCCAGTCTCCAGAGAAGGGGCTTGAGT
GGGTTGCTGAAATTAGAAACAAAGCTAATAATCATGCAACATACTATGC
TGAGTCTGTGAAAGGGAGGTTCACCATCTCAAGAGATGATTCCAAAAGG
AGAGTGTACCTGCAAATGAACACCTTAAGAGCTGAAGACACTGGCATTT
ATTACTGTACCGGGATCTACTATCATTACCCCTGGTTTGCTTACTGGGG
CCAAGGGACTCTGGTCACTGTCTCTGCAGCTAGCACCAAGGGCCCATCG
GTCTTCCCCCTGGCGCCCTGCTCCAGGAGCACCTCTGGGGGCACAGCGG
CCCTGGGCTGCCTGGTCAAGGACTACTTCCCCGAACCGGTGACGGTGTC
GTGGAACTCAGGCGCCCTGACCAGCGGCGTGCACACCTTCCCGGCTGTC
CTACAGTCCTCAGGACTCTACTCCCTCAGCAGCGTGGTGACCGTGCCCT
CCAGCAGCTTGGGCACCCAGACCTACACCTGCAACGTGAATCACAAGCC
CAGCAACACCAAGGTGGACAAGAGAGTTGAGCTCAAAACCCCACTTGGT
GACACAACTCACACATGCCCACGGTGCCCAGAGCCCAAATCTTGTGACA
CACCTCCCCCGTGCCCAAGGTGCCCAGAGCCCAAATCTTGTGACACACC
TCCCCCGTGCCCAAGGTGCCCAGAGCCCAAATCTTGTGACACACCTCCC
CCGTGCCCAAGGTGCCCAGCACCTGAACTCCTGGGAGGACCGTCAGTCT
TCCTCTTCCCCCCAAAACCCAAGGATACCCTTATGATTTCCCGGACCCC
TGAGGTCACGTGCGTGGTGGTGGACGTGAGCCACGAAGACCCCGAGGTC
CAGTTCAAGTGGTACGTGGACGGCGTGGAGGTGCATAATGCCAAGACAA
AGCTGCGGGAGGAGCAGTACAACAGCACGTTCCGTGTGGTCAGCGTCCT
CACCGTCCTGCACCAGGACTGGCTGAACGGCAAGGAGTACAAGTGCAAG
GTCTCCAACAAAGCCCTCCCAGCCCCCATCGAGAAAACCATCTCCAAAG
CCAAAGGACAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAACACCACGCCTCCCATGCTGGACTCCGACGGCTCCTT
CTTCCTCTACAGCAAGCTCACCGTGGACAAGAGCAGGTGGCAGCAGGGG
AACATCTTCTCATGCTCCGTGATGCATGAGGCTCTGCACAACCACTACA
CGCAGAAGAGCCTCTCCCTGTCTCCGGGTAAATCTGGTGGCGGTGGCTC
GGGCGGAGGTGGGTCGGGTGGCGGCGGATCCTGTGACCTGCCTCAGACT
CATAACCTCAGGAACAAGAGAGCCTTGACACTCCTGGTACAAATGAGGA
GACTCTCCCCTCTCTCCTGCCTGAAGGACAGGAAGGACTTTGGATTCCC
GCAGGAGAAGGTGGATGCCCAGCAGATCAAGAAGGCTCAAGCCATCCCT
GTCCTGAGTGAGCTGACCCAGCAGATCCTGAACATCTTCACATCAAAGG
ACTCATCTGCTGCTTGGAATGCAACCCTCCTAGACTCATTCTGCAATGA
CCTCCACCAGCAGCTCAATGACCTGCAAGGTTGTCTGATGCAGCAGGTG
GGGGTGCAGGAATTTCCCCTGACCCAGGAAGATGCCCTGCTGGCTGTGA
GGAAATACTTCCACAGGATCACTGTGTACCTGAGAGAGAAGAAACACAG
CCCCTGTGCCTGGGAGGTGGTCAGAGCAGAAGTCTGGAGAGCCCTGTCT
TCCTCTGCCAATGTGCTGGGAAGACTGAGAGAAGAGAAATG
Anti-DNS-IgG3-muIFNα long glyser linker-amino
acid sequence (SEQ ID NO: 2):
MYLGLNCVIIVFLLKGVQSEVKLEESGGGLVQPGGSMKLSCATSGFTFS
DAWMDWVRQSPEKGLEWVAEIRNKANNHATYYAESVKGRFTISRDDSKR
RVYLQMNTLRAEDTGIYYCTGIYYHYPWFAYWGQGTLVTVSAASTKGPS
VFPLAPCSRSTSGGTAALGCLVKDYFPEPVTVSWNSGALTSGVHTFPAV
LQSSGLYSLSSVVTVPSSSLGTQTYTCNVNHKPSNTKVDKRVELKTPLG
DTTHTCPRCPEPKSCDTPPPCPRCPEPKSCDTPPPCPRCPEPKSCDTPP
PCPRCPAPELLGGPSVFLFPPKPKDTLMISRTPEVTCVVVDVSHEDPEV
QFKWYVDGVEVHNAKTKLREEQYNSTFRVVSVLTVLHQDWLNGKEYKCK
VSNKALPAPIEKTISKAKGQPREPQVYTLPPSREEMTKNQVSLTCLVKG
FYPSDIAVEWESNGQPENNYNTTPPMLDSDGSFFLYSKLTVDKSRWQQG
NIFSCSVMHEALHNHYTQKSLSLSPGKSGGGGSGGGGSGGGGSCDLPQT
HNLRNKRALTLLVQMRRLSPLSCLKDRKDFGFPQEKVDAQQIKKAQAIP
VLSELTQQILNIFTSKDSSAAWNATLLDSFCNDLHQQLNDLQGCLMQQV
GVQEFPLTQEDALLAVRKYFHRITVYLREKKHSPCAWEVVRAEVWRALS
SSANVLGRLREEK
Nucleotide sequence of anti-DNS IgG3 GS1 human
IFN beta (SEQ ID NO: 3):
ATGTACTTGGGACTGAACTGTGTAATCATAGTTTTTCTCTTAAAAGGTG
TCCAGAGTGAAGTCAAGCTTGAGGAGTCTGGAGGAGGCTTGGTGCAACC
TGGAGGTTCCATGAAACTCTCTTGTGCTACTTCTGGATTCACTTTTAGT
GATGCCTGGATGGACTGGGTCCGCCAGTCTCCAGAGAAGGGGCTTGAGT
GGGTTGCTGAAATTAGAAACAAAGCTAATAATCATGCAACATACTATGC
TGAGTCTGTGAAAGGGAGGTTCACCATCTCAAGAGATGATTCCAAAAGG
AGAGTGTACCTGCAAATGAACACCTTAAGAGCTGAAGACACTGGCATTT
ATTACTGTACCGGGATCTACTATCATTACCCCTGGTTTGCTTACTGGGG
CCAAGGGACTCTGGTCACTGTCTCTGCAGCTAGCACCAAGGGCCCATCG
GTCTTCCCCCTGGCGCCCTGCTCCAGGAGCACCTCTGGGGGCACAGCGG
CCCTGGGCTGCCTGGTCAAGGACTACTTCCCCGAACCGGTGACGGTGTC
GTGGAACTCAGGCGCCCTGACCAGCGGCGTGCACACCTTCCCGGCTGTC
CTACAGTCCTCAGGACTCTACTCCCTCAGCAGCGTGGTGACCGTGCCCT
CCAGCAGCTTGGGCACCCAGACCTACACCTGCAACGTGAATCACAAGCC
CAGCAACACCAAGGTGGACAAGAGAGTTGAGCTCAAAACCCCACTTGGT
GACACAACTCACACATGCCCACGGTGCCCAGAGCCCAAATCTTGTGACA
CACCTCCCCCGTGCCCAAGGTGCCCAGAGCCCAAATCTTGTGACACACC
TCCCCCGTGCCCAAGGTGCCCAGAGCCCAAATCTTGTGACACACCTCCC
CCGTGCCCAAGGTGCCCAGCACCTGAACTCCTGGGAGGACCGTCAGTCT
TCCTCTTCCCCCCAAAACCCAAGGATACCCTTATGATTTCCCGGACCCC
TGAGGTCACGTGCGTGGTGGTGGACGTGAGCCACGAAGACCCCGAGGTC
CAGTTCAAGTGGTACGTGGACGGCGTGGAGGTGCATAATGCCAAGACAA
AGCTGCGGGAGGAGCAGTACAACAGCACGTTCCGTGTGGTCAGCGTCCT
CACCGTCCTGCACCAGGACTGGCTGAACGGCAAGGAGTACAAGTGCAAG
GTCTCCAACAAAGCCCTCCCAGCCCCCATCGAGAAAACCATCTCCAAAG
CCAAAGGACAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAACACCACGCCTCCCATGCTGGACTCCGACGGCTCCTT
CTTCCTCTACAGCAAGCTCACCGTGGACAAGAGCAGGTGGCAGCAGGGG
AACATCTTCTCATGCTCCGTGATGCATGAGGCTCTGCACAACCACTACA
CGCAGAAGAGCCTCTCCCTGTCTCCGGGTAAATCTGGTGGCGGTGGATC
CATGAGCTACAACTTGCTTGGATTCCTACAAAGAAGCAGCAATTTTCAG
TGTCAGAAGCTCCTGTGGCAATTGAATGGGAGGCTTGAATACTGCCTCA
AGGACAGGATGAACTTTGACATCCCTGAGGAGATTAAGCAGCTGCAGCA
GTTCCAGAAGGAGGACGCCGCATTGACCATCTATGAGATGCTCCAGAAC
ATCTTTGCTATTTTCAGACAAGATTCATCTAGCACTGGCTGGAATGAGA
CTATTGTTGAGAACCTCCTGGCTAATGTCTATCATCAGATAAACCATCT
GAAGACAGTCCTGGAAGAAAAACTGGAGAAAGAAGATTTCACCAGGGGA
AAACTCATGAGCAGTCTGCACCTGAAAAGATATTATGGGAGGATTCTGC
ATTACCTGAAGGCCAAGGAGTACAGTCACTGTGCCTGGACCATAGTCAG
AGTGGAAATCCTAAGGAACTTTTACTTCATTAACAGACTTACAGGTTAC
CTCCGAAACTGA
Amino acid sequence of anti-DNS IgG3 GS1 human
IFN beta (SEQ ID NO: 4):
M Y L G L N C V I I V F L L K G V Q S E V K L E E
S G G G L V Q P G G S M K L S C A T S G F T F S D
A W M D W V R Q S P E K G L E W V A E I R N K A N
N H A T Y Y A E S V K G R F T I S R D D S K R R V
Y L Q M N T L R A E D T G I Y Y C T G I Y Y H Y P
W F A Y W G Q G T L V T V S A A S T K G P S V F P
L A P C S R S T S G G T A A L G C L V K D Y F P E
P V T V S W N S G A L T S G V H T F P A V L Q S S
G L Y S L S S V V T V P S S S L G T Q T Y T C N V
N H K P S N T K V D K R V E L K T P L G D T T H T
C P R C P E P K S C D T P P P C P R C P E P K S C
D T P P P C P R C P E P K S C D T P P P C P R C P
A P E L L G G P S V F L F P P K P K D T L M I S R
T P E V T C V V V D V S H E D P E V Q F K W Y V D
G V E V H N A K T K L R E E Q Y N S T F R V V S V
L T V L H Q D W L N G K E Y K C K V S N K A L P A
P I E K T I S K A K G Q P R E P Q V Y T L P P S R
E E M T K N Q V S L T C L V K G F Y P S D I A V E
W E S N G Q P E N N Y N T T P P M L D S D G S F F
L Y S K L T V D K S R W Q Q G N I F S C S V M H E
A L H N H Y T Q K S L S L S P G K S G G G G S M S
Y N L L G F L Q R S S N F Q C Q K L L W Q L N G R
L E Y C L K D R M N F D I P E E I K Q L Q Q F Q K
E D A A L T I Y E M L Q N I F A I F R Q D S S S T
G W N E T I V E N L L A N V Y H Q I N H L K T V L
E E K L E K E D F T R G K L M S S L H L K R Y Y G
R I L H Y L K A K E Y S H C A W T I V R V E I L R
N F Y F I N R L T G Y L R N •
Nucleotide sequence of anti-DNS IgG3 GS1 murine
IFN beta (SEQ ID NO: 5):
ATGTACTTGGGACTGAACTGTGTAATCATAGTTTTTCTCTTAAAAGGTG
TCCAGAGTGAAGTCAAGCTTGAGGAGTCTGGAGGAGGCTTGGTGCAACC
TGGAGGTTCCATGAAACTCTCTTGTGCTACTTCTGGATTCACTTTTAGT
GATGCCTGGATGGACTGGGTCCGCCAGTCTCCAGAGAAGGGGCTTGAGT
GGGTTGCTGAAATTAGAAACAAAGCTAATAATCATGCAACATACTATGC
TGAGTCTGTGAAAGGGAGGTTCACCATCTCAAGAGATGATTCCAAAAGG
AGAGTGTACCTGCAAATGAACACCTTAAGAGCTGAAGACACTGGCATTT
ATTACTGTACCGGGATCTACTATCATTACCCCTGGTTTGCTTACTGGGG
CCAAGGGACTCTGGTCACTGTCTCTGCAGCTAGCACCAAGGGCCCATCG
GTCTTCCCCCTGGCGCCCTGCTCCAGGAGCACCTCTGGGGGCACAGCGG
CCCTGGGCTGCCTGGTCAAGGACTACTTCCCCGAACCGGTGACGGTGTC
GTGGAACTCAGGCGCCCTGACCAGCGGCGTGCACACCTTCCCGGCTGTC
CTACAGTCCTCAGGACTCTACTCCCTCAGCAGCGTGGTGACCGTGCCCT
CCAGCAGCTTGGGCACCCAGACCTACACCTGCAACGTGAATCACAAGCC
CAGCAACACCAAGGTGGACAAGAGAGTTGAGCTCAAAACCCCACTTGGT
GACACAACTCACACATGCCCACGGTGCCCAGAGCCCAAATCTTGTGACA
CACCTCCCCCGTGCCCAAGGTGCCCAGAGCCCAAATCTTGTGACACACC
TCCCCCGTGCCCAAGGTGCCCAGAGCCCAAATCTTGTGACACACCTCCC
CCGTGCCCAAGGTGCCCAGCACCTGAACTCCTGGGAGGACCGTCAGTCT
TCCTCTTCCCCCCAAAACCCAAGGATACCCTTATGATTTCCCGGACCCC
TGAGGTCACGTGCGTGGTGGTGGACGTGAGCCACGAAGACCCCGAGGTC
CAGTTCAAGTGGTACGTGGACGGCGTGGAGGTGCATAATGCCAAGACAA
AGCTGCGGGAGGAGCAGTACAACAGCACGTTCCGTGTGGTCAGCGTCCT
CACCGTCCTGCACCAGGACTGGCTGAACGGCAAGGAGTACAAGTGCAAG
GTCTCCAACAAAGCCCTCCCAGCCCCCATCGAGAAAACCATCTCCAAAG
CCAAAGGACAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAACACCACGCCTCCCATGCTGGACTCCGACGGCTCCTT
CTTCCTCTACAGCAAGCTCACCGTGGACAAGAGCAGGTGGCAGCAGGGG
AACATCTTCTCATGCTCCGTGATGCATGAGGCTCTGCACAACCACTACA
CGCAGAAGAGCCTCTCCCTGTCTCCGGGTAAATCTGGTGGCGGTGGATC
CATCAACTATAAGCAGCTCCAGCTCCAAGAAAGGACGAACATTCGGAAA
TGTCAGGAGCTCCTGGAGCAGCTGAATGGAAAGATCAACCTCACCTACA
GGGCGGACTTTAAGATCCCTATGGAGATGACGGAGAAGATGCAGAAGAG
TTACACTGCCTTTGCCATCCAAGAGATGCTCCAGAATGTCTTTCTTGTC
TTCAGAAACAATTTCTCCAGCACTGGGTGGAATGAGACTATTGTTGTAC
GTCTCCTGGATGAACTCCACCAGCAGACAGTGTTTCTGAAGACAGTACT
AGAGGAAAAGCAAGAGGAAAGATTGACGTGGGAGATGTCCTCAACTGCT
CTCCACTTGAAGAGCTATTACTGGAGGGTGCAAAGGTACCTTAAACTCA
TGAAGTACAACAGCTACGCCTGGATGGTGGTCCGAGCAGAGATCTTCAG
GAACTTTCTCATCATTCGAAGACTTACCAGAAACTTCCAAAACTGA
Amino acid sequence of anti-DNS IgG3 GS1 murine
IFN beta (SEQ ID NO: 6):
M Y L G L N C V I I V F L L K G V Q S E V K L E E
S G G G L V Q P G G S M K L S C A T S G F T F S D
A W M D W V R Q S P E K G L E W V A E I R N K A N
N H A T Y Y A E S V K G R F T I S R D D S K R R V
Y L Q M N T L R A E D T G I Y Y C T G I Y Y H Y P
W F A Y W G Q G T L V T V S A A S T K G P S V F P
L A P C S R S T S G G T A A L G C L V K D Y F P E
P V T V S W N S G A L T S G V H T F P A V L Q S S
G L Y S L S S V V T V P S S S L G T Q T Y T C N V
N H K P S N T K V D K R V E L K T P L G D T T H T
C P R C P E P K S C D T P P P C P R C P E P K S C
D T P P P C P R C P E P K S C D T P P P C P R C P
A P E L L G G P S V F L F P P K P K D T L M I S R
T P E V T C V V V D V S H E D P E V Q F K W Y V D
G V E V H N A K T K L R E E Q Y N S T F R V V S V
L T V L H Q D W L N G K E Y K C K V S N K A L P A
P I E K T I S K A K G Q P R E P Q V Y T L P P S R
E E M T K N Q V S L T C L V K G F Y P S D I A V E
W E S N G Q P E N N Y N T T P P M L D S D G S F F
L Y S K L T V D K S R W Q Q G N I F S C S V M H E
A L H N H Y T Q K S L S L S P G K S G G G G S I N
Y K Q L Q L Q E R T N I R K C Q E L L E Q L N G K
I N L T Y R A D F K I P M E M T E K M Q K S Y T A
F A I Q E M L Q N V F L V F R N N F S S T G W N E
T I V V R L L D E L H Q Q T V F L K T V L E E K Q
E E R L T W E M S S T A L H L K S Y Y W R V Q R Y
L K L M K Y N S Y A W M V V R A E I F R N F L I I
R R L T R N F Q N •
Anti-DNS-IgG3-muIFNα glyser linker-nucleic acid
sequence (SEQ ID NO: 7):
ATGTACTTGGGACTGAACTGTGTAATCATAGTTTTTCTCTTAAAAGGTG
TCCAGAGTGAAGTCAAGCTTGAGGAGTCTGGAGGAGGCTTGGTGCAACC
TGGAGGTTCCATGAAACTCTCTTGTGCTACTTCTGGATTCACTTTTAGT
GATGCCTGGATGGACTGGGTCCGCCAGTCTCCAGAGAAGGGGCTTGAGT
GGGTTGCTGAAATTAGAAACAAAGCTAATAATCATGCAACATACTATGC
TGAGTCTGTGAAAGGGAGGTTCACCATCTCAAGAGATGATTCCAAAAGG
AGAGTGTACCTGCAAATGAACACCTTAAGAGCTGAAGACACTGGCATTT
ATTACTGTACCGGGATCTACTATCATTACCCCTGGTTTGCTTACTGGGG
CCAAGGGACTCTGGTCACTGTCTCTGCAGCTAGCACCAAGGGCCCATCG
GTCTTCCCCCTGGCGCCCTGCTCCAGGAGCACCTCTGGGGGCACAGCGG
CCCTGGGCTGCCTGGTCAAGGACTACTTCCCCGAACCGGTGACGGTGTC
GTGGAACTCAGGCGCCCTGACCAGCGGCGTGCACACCTTCCCGGCTGTC
CTACAGTCCTCAGGACTCTACTCCCTCAGCAGCGTGGTGACCGTGCCCT
CCAGCAGCTTGGGCACCCAGACCTACACCTGCAACGTGAATCACAAGCC
CAGCAACACCAAGGTGGACAAGAGAGTTGAGCTCAAAACCCCACTTGGT
GACACAACTCACACATGCCCACGGTGCCCAGAGCCCAAATCTTGTGACA
CACCTCCCCCGTGCCCAAGGTGCCCAGAGCCCAAATCTTGTGACACACC
TCCCCCGTGCCCAAGGTGCCCAGAGCCCAAATCTTGTGACACACCTCCC
CCGTGCCCAAGGTGCCCAGCACCTGAACTCCTGGGAGGACCGTCAGTCT
TCCTCTTCCCCCCAAAACCCAAGGATACCCTTATGATTTCCCGGACCCC
TGAGGTCACGTGCGTGGTGGTGGACGTGAGCCACGAAGACCCCGAGGTC
CAGTTCAAGTGGTACGTGGACGGCGTGGAGGTGCATAATGCCAAGACAA
AGCTGCGGGAGGAGCAGTACAACAGCACGTTCCGTGTGGTCAGCGTCCT
CACCGTCCTGCACCAGGACTGGCTGAACGGCAAGGAGTACAAGTGCAAG
GTCTCCAACAAAGCCCTCCCAGCCCCCATCGAGAAAACCATCTCCAAAG
CCAAAGGACAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAACACCACGCCTCCCATGCTGGACTCCGACGGCTCCTT
CTTCCTCTACAGCAAGCTCACCGTGGACAAGAGCAGGTGGCAGCAGGGG
AACATCTTCTCATGCTCCGTGATGCATGAGGCTCTGCACAACCACTACA
CGCAGAAGAGCCTCTCCCTGTCTCCGGGTAAATCTGGTGGCGGTGGATC
CTGTGACCTGCCTCAGACTCATAACCTCAGGAACAAGAGAGCCTTGACA
CTCCTGGTACAAATGAGGAGACTCTCCCCTCTCTCCTGCCTGAAGGACA
GGAAGGACTTTGGATTCCCGCAGGAGAAGGTGGATGCCCAGCAGATCAA
GAAGGCTCAAGCCATCCCTGTCCTGAGTGAGCTGACCCAGCAGATCCTG
AACATCTTCACATCAAAGGACTCATCTGCTGCTTGGAATGCAACCCTCC
TAGACTCATTCTGCAATGACCTCCACCAGCAGCTCAATGACCTGCAAGG
TTGTCTGATGCAGCAGGTGGGGGTGCAGGAATTTCCCCTGACCCAGGAA
GATGCCCTGCTGGCTGTGAGGAAATACTTCCACAGGATCACTGTGTACC
TGAGAGAGAAGAAACACAGCCCCTGTGCCTGGGAGGTGGTCAGAGCAGA
AGTCTGGAGAGCCCTGTCTTCCTCTGCCAATGTGCTGGGAAGACTGAGA
GAAGAGAAA
Anti-DNS-IgG3-muIFNα glyser linker-amino acid
sequence (SEQ ID NO: 8):
MYLGLNCVIIVFLLKGVQSEVKLEESGGGLVQPGGSMKLSCATSGFTFS
DAWMDWVRQSPEKGLEWVAEIRNKANNHATYYAESVKGRFTISRDDSKR
RVYLQMNTLRAEDTGIYYCTGIYYHYPWFAYWGQGTLVTVSAASTKGPS
VFPLAPCSRSTSGGTAALGCLVKDYFPEPVTVSWNSGALTSGVHTFPAV
LQSSGLYSLSSVVTVPSSSLGTQTYTCNVNHKPSNTKVDKRVELKTPLG
DTTHTCPRCPEPKSCDTPPPCPRCPEPKSCDTPPPCPRCPEPKSCDTPP
PCPRCPAPELLGGPSVFLFPPKPKDTLMISRTPEVTCVVVDVSHEDPEV
QFKWYVDGVEVHNAKTKLREEQYNSTFRVVSVLTVLHQDWLNGKEYKCK
VSNKALPAPIEKTISKAKGQPREPQVYTLPPSREEMTKNQVSLTCLVKG
FYPSDIAVEWESNGQPENNYNTTPPMLDSDGSFFLYSKLTVDKSRWQQG
NIFSCSVMHEALHNHYTQKSLSLSPGKSGGGGSCDLPQTHNLRNKRALT
LLVQMRRLSPLSCLKDRKDFGFPQEKVDAQQIKKAQAIPVLSELTQQIL
NIFTSKDSSAAWNATLLDSFCNDLHQQLNDLQGCLMQQVGVQEFPLTQE
DALLAVRKYFHRITVYLREKKHSPCAWEVVRAEVWRALSSSANVLGRLR
EEK

### Example 3

### Useful Nucleic Acid and Protein Sequences for Use According to the Invention

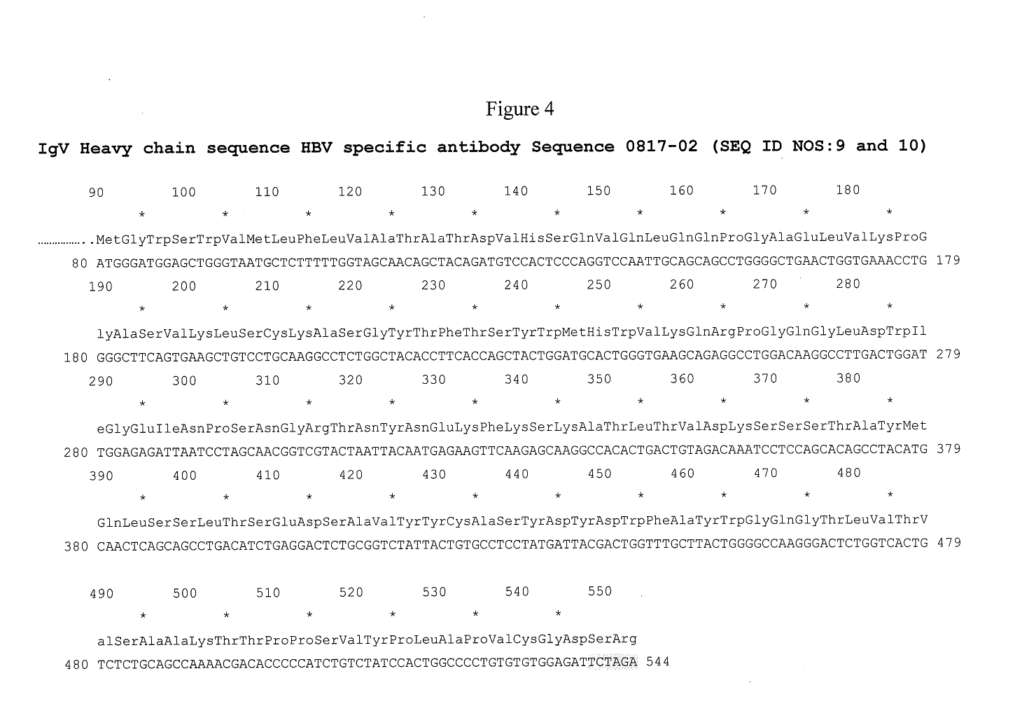

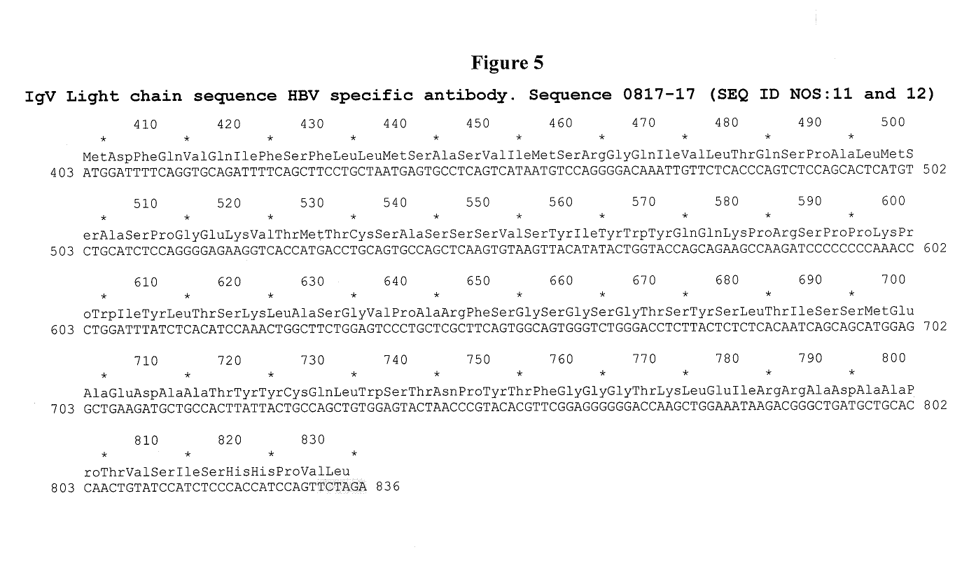

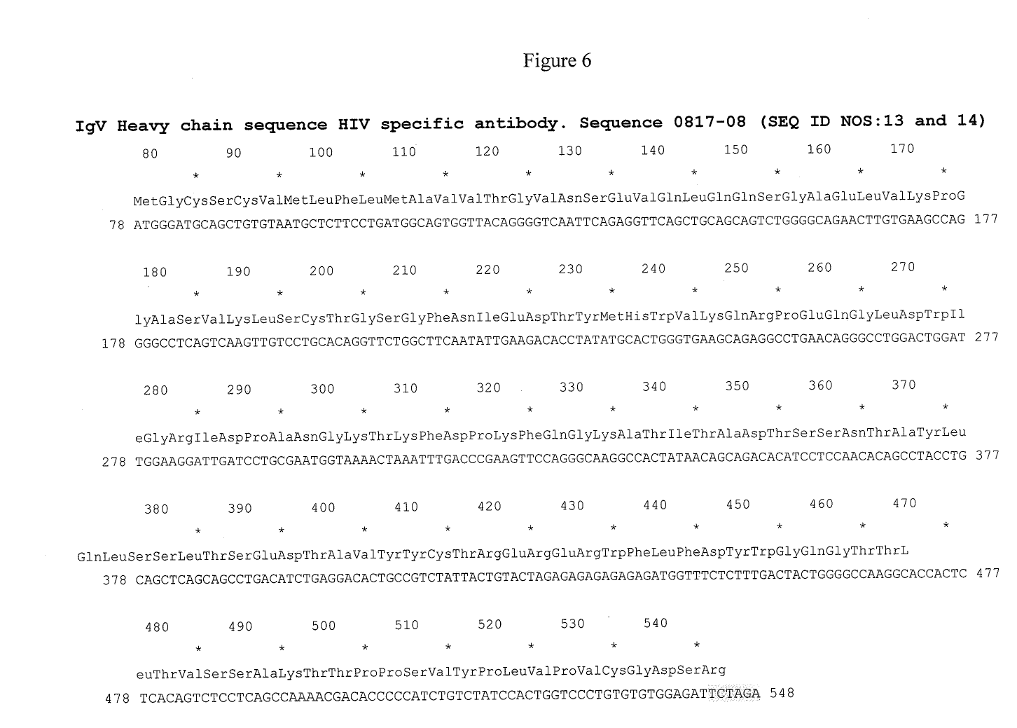

Anti-viral antibodies for use according to the invention are set forth in FIGS. 4 to 6.

Suitable human interferon beta sequences (SEQ ID NO:15) includes mtnkcllqia lllcfsttal smsynllgfl qrssncqcqk llwqlngrle yclkdlinfdipeeikqlqq fqkedaavti yemlqnifai frqdssstgw netivenlla nvyhqrnhlktvleekleke dftrgkrmss lhlkryygri lhylkakeds hcawtivrve ilrnfyvinrltgylrn (GenBank: AAC41702.1)

Suitable human interferon alpha sequences (SEQ ID NOS:16 and 17) include: mallfpllaa lvmtsyspvg slgcdlpqnh gllsrntivl lhqmrrispf lclkdrrdfrfpqemvkgsq lqkahvmsvl hemlqqifsl fhterssaaw nmtlldqlht elhqqlqhletcllqvvgeg esagaisspa ltlrryfqgi rvylkekkys dcawevvrme imkslflstnmqerlrskdr dlgss (GenBank: AAA52724.1) maltfyllva lvvlsyksfs slgedlpqth signrralil laqmrrispf sclkdrhdfefpqeefddkq fqkaqaisvl hemiqqtfnl fstkdssaal detlldefyi eldqqlndlescvmqevgvi esplmyedsi lavrkyfqri tlyltekkys scawevvrae imrsfslsin lqkrlkske (GenBank: AAA52716.1)

### Example 4

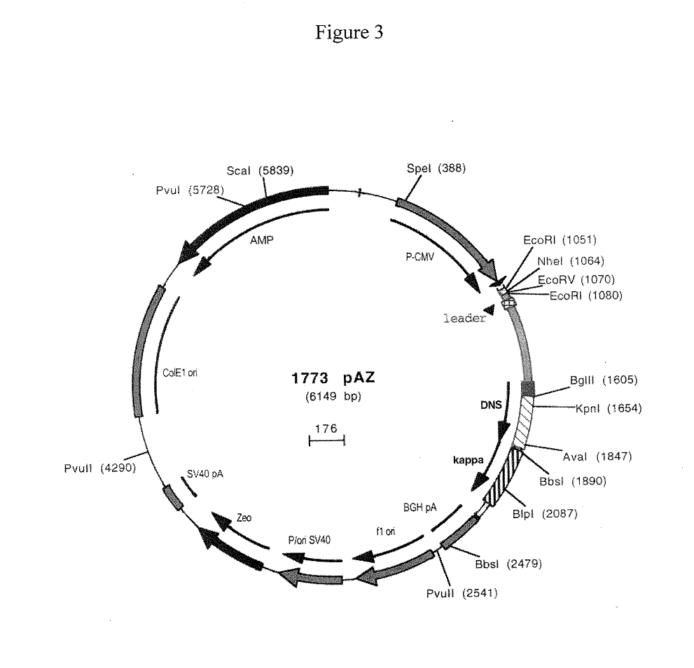

Murine NIH 3T3 cells were contacted with recombinant murine IFN-β alone or conjugated with an IgG. Human 3T3 cells were contact with an IgG-human IFN-β and the effects of the protein on the immune activation of the cells assessed. The results are shown in FIG. 3.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of treating a virus infection in a subject, said method comprising administering a fusion protein to the subject, wherein the fusion protein comprises a cytokine fused to an antibody or a fragment of the antibody which binds to a cell infected by the virus.

2. The method of claim 1, wherein the cell is a hepatocyte and the antibody binds to a cell surface antigen of the hepatocyte.

3. The method of claim 1, wherein the cytokine is an interferon α or interferon β.

4. The method of claim 1, wherein the subject and the cytokine belong to the same species.

5. The method of claim 4, wherein the subject is human.

6. The method of claim 1, wherein the cytokine is fused to a C-terminus of the antibody.

7. The method of claim 1, wherein the antibody is IgG.

8. The method of claim 1, wherein the virus is HCV, HBV, HSV, HPV, or HIV and the subject is human.

9. The method of claim 1, wherein the antibody is a minibody or a diabody.

10. The method of claim 1, wherein the antibody or fragment thereof recognizes an epitope of the virus.

11. The method of claim 1, wherein the antibody or fragment thereof recognizes a constituent of the cell surface.

12. The method of claim 1, wherein the antibody or fragment thereof recognizes a cell surface receptor or antigen of the target cell

13. The method of claim 1, wherein the antibody or fragment thereof targets a viral antigen expressed on the surface of a cell or in a tissue infected with the virus.

14. A method of modulating an immune response in a subject, said method comprising administering to the subject a fusion protein comprising a cytokine fused to an antibody which binds to a target cell of the immune system.

15. The method of claim 14, wherein the cytokine is an interferon and the target cell is a Th17 cell of the mouse or the human equivalent.

16. The method of claim 14, wherein an autoimmune condition of the subject is treated.

17. The method of claim 14, wherein the autoimmune condition is Multiple sclerosis, myasthenia gravis, asthma, allergy, IBD or colitis, rheumatoid arthritis, Graves disease, or Type I diabetes.

18. The method of claim 16, wherein the interferon is interferon β or α.

19. The method of claim 16, wherein the interferon is a hybrid interferon.

20. The method of claim 14, wherein the subject is human.


SEQUENCE LISTING (17 sequences)

SEQ ID NO: 1 - DNA, 2099 bp, Artificial Sequence
  synthetic anti-DNS-IgG3-muIFNalpha long GlySer linker
atgtacttgggactgaactgtgtaatcatagtttttctcttaaaaggtgtccagagtgaagtcaagcttgaggagtctggaggaggcttggtgcaacctggaggttccatgaaactctcttgtgctacttctggattcacttttagtgatgcctggatggactgggtccgccagtctccagagaaggggcttgagtgggttgctgaaattagaaacaaagctaataatcatgcaacatactatgctgagtctgtgaaagggaggttcaccatctcaagagatgattccaaaaggagagtgtacctgcaaatgaacaccttaagagctgaagacactggcatttattactgtaccgggatctactatcattacccctggtttgcttactggggccaagggactctggtcactgtctctgcagctagcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccaaggtgcccagagcccaaatcttgtgacacacctcccccgtgcccaaggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagctgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaagccaaaggacagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaatctggtggcggtggctcgggcggaggtgggtcgggtggcggcggatcctgtgacctgcctcagactcataacctcaggaacaagagagccttgacactcctggtacaaatgaggagactctcccctctctcctgcctgaaggacaggaaggactttggattcccgcaggagaaggtggatgcccagcagatcaagaaggctcaagccatccctgtcctgagtgagctgacccagcagatcctgaacatcttcacatcaaaggactcatctgctgcttggaatgcaaccctcctagactcattctgcaatgacctccaccagcagctcaatgacctgcaaggttgtctgatgcagcaggtgggggtgcaggaatttcccctgacccaggaagatgccctgctggctgtgaggaaatacttccacaggatcactgtgtacctgagagagaagaaacacagcccctgtgcctgggaggtggtcagagcagaagtctggagagccctgtcttcctctgccaatgtgctgggaagactgagagaagagaaatg

SEQ ID NO: 2 - protein, 699 aa, Artificial Sequence
  synthetic anti-DNS-IgG3-muIFNalpha long GlySer linker
MYLGLNCVIIVFLLKGVQSEVKLEESGGGLVQPGGSMKLSCATSGFTFSDAWMDWVRQSPEKGLEWVAEIRNKANNHATYYAESVKGRFTISRDDSKRRVYLQMNTLRAEDTGIYYCTGIYYHYPWFAYWGQGTLVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKAKGQPREPQVYTLPPSREEMTKNQVSLTCLVKGFYPSDIAVEWESNGQPENNYNTTPPMLDSDGSFFLYSKLTVDKSRWQQGNIFSCSVMHEALHNHYTQKSLSLSPGKSGGGGSGGGGSGGGGSCDLPQTHNLRNKRALTLLVQMRRLSPLSCLKDRKDFGFPQEKVDAQQIKKAQAIPVLSELTQQILNIFTSKDSSAAWNATLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 3 - DNA, 2070 bp, Artificial Sequence
  synthetic anti-DNS IgG3 GS1 human IFN beta
atgtacttgggactgaactgtgtaatcatagtttttctcttaaaaggtgtccagagtgaagtcaagcttgaggagtctggaggaggcttggtgcaacctggaggttccatgaaactctcttgtgctacttctggattcacttttagtgatgcctggatggactgggtccgccagtctccagagaaggggcttgagtgggttgctgaaattagaaacaaagctaataatcatgcaacatactatgctgagtctgtgaaagggaggttcaccatctcaagagatgattccaaaaggagagtgtacctgcaaatgaacaccttaagagctgaagacactggcatttattactgtaccgggatctactatcattacccctggtttgcttactggggccaagggactctggtcactgtctctgcagctagcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccaaggtgcccagagcccaaatcttgtgacacacctcccccgtgcccaaggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagctgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaagccaaaggacagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaatctggtggcggtggatccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 4 - protein, 689 aa, Artificial Sequence
  synthetic anti-DNS IgG3 GS1 human IFN beta
MYLGLNCVIIVFLLKGVQSEVKLEESGGGLVQPGGSMKLSCATSGFTFSDAWMDWVRQSPEKGLEWVAEIRNKANNHATYYAESVKGRFTISRDDSKRRVYLQMNTLRAEDTGIYYCTGIYYHYPWFAYWGQGTLVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKAKGQPREPQVYTLPPSREEMTKNQVSLTCLVKGFYPSDIAVEWESNGQPENNYNTTPPMLDSDGSFFLYSKLTVDKSRWQQGNIFSCSVMHEALHNHYTQKSLSLSPGKSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 5 - DNA, 2055 bp, Artificial Sequence
  synthetic anti-DNS IgG3 GS1 murine IFN beta
atgtacttgggactgaactgtgtaatcatagtttttctcttaaaaggtgtccagagtgaagtcaagcttgaggagtctggaggaggcttggtgcaacctggaggttccatgaaactctcttgtgctacttctggattcacttttagtgatgcctggatggactgggtccgccagtctccagagaaggggcttgagtgggttgctgaaattagaaacaaagctaataatcatgcaacatactatgctgagtctgtgaaagggaggttcaccatctcaagagatgattccaaaaggagagtgtacctgcaaatgaacaccttaagagctgaagacactggcatttattactgtaccgggatctactatcattacccctggtttgcttactggggccaagggactctggtcactgtctctgcagctagcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccaaggtgcccagagcccaaatcttgtgacacacctcccccgtgcccaaggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagctgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaagccaaaggacagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaatctggtggcggtggatccatcaactataagcagctccagctccaagaaaggacgaacattcggaaatgtcaggagctcctggagcagctgaatggaaagatcaacctcacctacagggcggactttaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactga

SEQ ID NO: 6 - protein, 684 aa, Artificial Sequence
  synthetic anti-DNS IgG3 GS1 murine IFN beta
MYLGLNCVIIVFLLKGVQSEVKLEESGGGLVQPGGSMKLSCATSGFTFSDAWMDWVRQSPEKGLEWVAEIRNKANNHATYYAESVKGRFTISRDDSKRRVYLQMNTLRAEDTGIYYCTGIYYHYPWFAYWGQGTLVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKAKGQPREPQVYTLPPSREEMTKNQVSLTCLVKGFYPSDIAVEWESNGQPENNYNTTPPMLDSDGSFFLYSKLTVDKSRWQQGNIFSCSVMHEALHNHYTQKSLSLSPGKSGGGGSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 7 - DNA, 2067 bp, Artificial Sequence
  synthetic anti-DNS-IgG3-muIFNalpha GlySer linker
atgtacttgggactgaactgtgtaatcatagtttttctcttaaaaggtgtccagagtgaagtcaagcttgaggagtctggaggaggcttggtgcaacctggaggttccatgaaactctcttgtgctacttctggattcacttttagtgatgcctggatggactgggtccgccagtctccagagaaggggcttgagtgggttgctgaaattagaaacaaagctaataatcatgcaacatactatgctgagtctgtgaaagggaggttcaccatctcaagagatgattccaaaaggagagtgtacctgcaaatgaacaccttaagagctgaagacactggcatttattactgtaccgggatctactatcattacccctggtttgcttactggggccaagggactctggtcactgtctctgcagctagcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccaaggtgcccagagcccaaatcttgtgacacacctcccccgtgcccaaggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagctgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaagccaaaggacagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaatctggtggcggtggatcctgtgacctgcctcagactcataacctcaggaacaagagagccttgacactcctggtacaaatgaggagactctcccctctctcctgcctgaaggacaggaaggactttggattcccgcaggagaaggtggatgcccagcagatcaagaaggctcaagccatccctgtcctgagtgagctgacccagcagatcctgaacatcttcacatcaaaggactcatctgctgcttggaatgcaaccctcctagactcattctgcaatgacctccaccagcagctcaatgacctgcaaggttgtctgatgcagcaggtgggggtgcaggaatttcccctgacccaggaagatgccctgctggctgtgaggaaatacttccacaggatcactgtgtacctgagagagaagaaacacagcccctgtgcctgggaggtggtcagagcagaagtctggagagccctgtcttcctctgccaatgtgctgggaagactgagagaagagaaa

SEQ ID NO: 8 - protein, 689 aa, Artificial Sequence
  synthetic anti-DNS-IgG3-muIFNalpha GlySer linker
MYLGLNCVIIVFLLKGVQSEVKLEESGGGLVQPGGSMKLSCATSGFTFSDAWMDWVRQSPEKGLEWVAEIRNKANNHATYYAESVKGRFTISRDDSKRRVYLQMNTLRAEDTGIYYCTGIYYHYPWFAYWGQGTLVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKAKGQPREPQVYTLPPSREEMTKNQVSLTCLVKGFYPSDIAVEWESNGQPENNYNTTPPMLDSDGSFFLYSKLTVDKSRWQQGNIFSCSVMHEALHNHYTQKSLSLSPGKSGGGGSCDLPQTHNLRNKRALTLLVQMRRLSPLSCLKDRKDFGFPQEKVDAQQIKKAQAIPVLSELTQQILNIFTSKDSSAAWNATLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 9 - DNA, 465 bp, Artificial Sequence
  synthetic anti-HBV IgV heavy chain, Sequence 0817-02
atgggatggagctgggtaatgctctttttggtagcaacagctacagatgtccactcccaggtccaattgcagcagcctggggctgaactggtgaaacctggggcttcagtgaagctgtcctgcaaggcctctggctacaccttcaccagctactggatgcactgggtgaagcagaggcctggacaaggccttgactggattggagagattaatcctagcaacggtcgtactaattacaatgagaagttcaagagcaaggccacactgactgtagacaaatcctccagcacagcctacatgcaactcagcagcctgacatctgaggactctgcggtctattactgtgcctcctatgattacgactggtttgcttactggggccaagggactctggtcactgtctctgcagccaaaacgacacccccatctgtctatccactggcccctgtgtgtggagattctaga

SEQ ID NO: 10 - protein, 155 aa, Artificial Sequence
  synthetic anti-HBV IgV heavy chain, Sequence 0817-02
MGWSWVMLFLVATATDVHSQVQLQQPGAELVKPGASVKLSCKASGYTFTSYWMHWVKQRPGQGLDWIGEINPSNGRTNYNEKFKSKATLTVDKSSSTAYMQLSSLTSEDSAVYYCASYDYDWFAYWGQGTLVTVSAAKTTPPSVYPLAPVCGDSR

SEQ ID NO: 11 - DNA, 434 bp, Artificial Sequence
  synthetic anti-HBV IgV light chain, Sequence 0817-17
atggattttcaggtgcagattttcagcttcctgctaatgagtgcctcagtcataatgtccaggggacaaattgttctcacccagtctccagcactcatgtctgcatctccaggggagaaggtcaccatgacctgcagtgccagctcaagtgtaagttacatatactggtaccagcagaagccaagatccccccccaaaccctggatttatctcacatccaaactggcttctggagtccctgctcgcttcagtggcagtgggtctgggacctcttactctctcacaatcagcagcatggaggctgaagatgctgccacttattactgccagctgtggagtactaacccgtacacgttcggaggggggaccaagctggaaataagacgggctgatgctgcaccaactgtatccatctcccaccatccagttctaga

SEQ ID NO: 12 - protein, 144 aa, Artificial Sequence
  synthetic anti-HBV IgV light chain, Sequence 0817-17
MDFQVQIFSFLLMSASVIMSRGQIVLTQSPALMSASPGEKVTMTCSASSSVSYIYWYQQKPRSPPKPWIYLTSKLASGVPARFSGSGSGTSYSLTISSMEAEDAATYYCQLWSTNPYTFGGGTKLEIRRADAAPTVSISHHPVL

SEQ ID NO: 13 - DNA, 471 bp, Artificial Sequence
  synthetic anti-HIV IgV heavy chain, Sequence 0817-08
atgggatgcagctgtgtaatgctcttcctgatggcagtggttacaggggtcaattcagaggttcagctgcagcagtctggggcagaacttgtgaagccaggggcctcagtcaagttgtcctgcacaggttctggcttcaatattgaagacacctatatgcactgggtgaagcagaggcctgaacagggcctggactggattggaaggattgatcctgcgaatggtaaaactaaatttgacccgaagttccagggcaaggccactataacagcagacacatcctccaacacagcctacctgcagctcagcagcctgacatctgaggacactgccgtctattactgtactagagagagagagagatggtttctctttgactactggggccaaggcaccactctcacagtctcctcagccaaaacgacacccccatctgtctatccactggtccctgtgtgtggagattctaga

SEQ ID NO: 14 - protein, 157 aa, Artificial Sequence
  synthetic anti-HIV IgV heavy chain, Sequence 0817-08
MGCSCVMLFLMAVVTGVNSEVQLQQSGAELVKPGASVKLSCTGSGFNIEDTYMHWVKQRPEQGLDWIGRIDPANGKTKFDPKFQGKATITADTSSNTAYLQLSSLTSEDTAVYYCTRERERWFLFDYWGQGTTLTVSSAKTTPPSVYPLVPVCGDSR

SEQ ID NO: 15 - protein, 187 aa, Homo sapiens [sequence omitted]

SEQ ID NO: 16 - protein, 195 aa, Homo sapiens [sequence omitted]

SEQ ID NO: 17 - protein, 189 aa, Homo sapiens
  human interferon alpha, type 201 from leukocyte (IFNalpha, IFNA, HUMIFNAAP)
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE